(19) United States
(12) Patent Application Publication
(10) Pub. No.: US 2025/0288352 A1
(43) Pub. Date: Sep. 18, 2025

(54) COMBINED RF FRACTIONAL AND NON-FRACTIONAL TREATMENT

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 18/604,694

(22) Filed: Mar. 14, 2024

(51) Int. Cl. A61B 18/20; A61B 18/00

(52) U.S. Cl. CPC A61B 18/20; A61B 2018/00458; A61B 2018/00577; A61B 2018/2005; A61B 2018/20351

(57) ABSTRACT

A method for fractional tissue treatment includes using a first energy source to heat a skin treatment zone to a sub-necrotic temperature level, and using a second energy source to apply fractional energy to the skin treatment zone to create a matrix of ablation and/or coagulation micro-zones.

### FIELD OF THE INVENTION

The invention relates to a device and method for fractional laser treatment of human tissue combined with non-fractional treatment of a treated zone.

### BACKGROUND OF THE INVENTION

Fractional devices are known for skin treatment. Fractional injuries to the skin and dermis can be delivered by laser systems emitting small beams of laser radiation into the skin or alternatively fractional devices, such as micro-needling, surface ablation or invasive needling. The advantage of segmental, fractional injury is the dermis is stimulated with an aggressive fractional trauma providing fractional skin resurfacing, skin tightening, acne scar and wrinkle treatment as well as treatment of hyperhidrosis, acne and transdermal drug delivery.

U.S. Pat. No. 6,210,402 describes a method for dermatological treatment of an external body surface by applying high frequency electrical energy to an electrode terminal comprising multiple conductive elements.

U.S. Pat. Nos. 6,148,232 and 6,615,079 describe methods and devices for fractional ablation of the stratum corneum for transdermal drug delivery. Conductive elements are applied to the stratum corneum and RF energy is applied between the conductive elements.

U.S. Pat. Nos. 8,496,654 and 8,357,157 describe devices for cosmetic fractional epidermis ablation. Multiple electrodes are applied to the skin surface and RF energy is applied between the electrodes and a grounded return electrode. The RF application elements are free of any ground electrode therebetween.

U.S. Pat. No. 9,480,836 describes a needle array penetrating into the skin and powered by a motor connecting to the array. RF energy is applied between needles penetrating into the skin.

All the above mentioned inventions describe devices that create a non-uniform thermal effect around needles or pins in a matrix of electrodes. Designs in which RF energy is applied between a cluster of pins and a large return electrode are very sensitive to the position of the pin in the cluster. Some pins have a large distance from the return electrode while others are very close, which creates non-equal thermal effects around different pins in the matrix.

U.S. Pat. No. 10,973,578 describes a laser system configured to emit at least one laser beam, and an optical system configured to focus the laser beam(s) to a focal region at a selected distance from a surface of a tissue. The focal region can be configured to illuminate at least a portion of a target.

U.S. Pat. No. 8,286,640 describes a method for fractional dermatological treatment, including directing an optical beam emitted by a laser source toward a target region of skin to deliver a fractional treatment to the target region of skin; The invention describes a method for fractional heating of the skin up to 80° C.

Collagen remodeling and skin rejuvenation are aggressive treatments. Deeper ablation and coagulation of the skin combined with high density of ablation zones increase treatment efficiency, but are associated with stronger pain, longer down time and higher risk of side effects including post-inflammatory hyper-pigmentation and skin discoloration.

### SUMMARY

The present invention seeks to provide a method and device for skin remodeling creating thermal inflammation of the treatment zone, by applying energy penetrating into the skin and elevating skin temperature to a sub-necrotic level combined with essentially simultaneous fractional skin ablation/coagulation. The skin heating to the sub-necrotic level can be done by using infrared (IR) radiation produced by laser, power light emitted diodes (LED) or VCSEL (vertical-cavity surface-emitting laser). Alternatively, heating can be done by using a filament lamp or a gas discharge lamp. The invention has improved efficiency compared to the prior art, with minimal ablation/coagulation of the skin.

Other types of energy including ultrasound, radio-frequency (RF) and microwave can be used for non-selective heating of tissue.

The tissue can be heated prior to the fractional ablation, simultaneously or shortly after the non-selective bulk heating of the skin.

Contact heating also can be used but it takes longer to create uniform heating over the skin depth.

The bulk heating of the tissue may include heating to a sub-necrotic level of about 40-47° C. Skin heating to this temperature cause skin inflammation, appearing as redness and swelling. The heating should be through the depth required to be remodeled, which typically varies from about 0.6 mm in the periorbital area up to 3 mm in other body areas. For treating the subdermal zone, the depth can be extended to 5 mm and deeper.

Optical energy in the near infrared range from 700 nm to 1900 nm can be used to create bulk uniform heating of the skin. Longer wavelengths have strong absorption in water and are more suitable for tissue ablation and coagulation. Shorter wavelengths have strong selective absorption in hemoglobin and melanin and penetrate to a lower depth of the skin.

RF energy is also a good alternative for the bulk heating of the skin. Tissue can be heated with monopolar or bi-polar RF electrodes delivering RF energy to the skin.

Heating of the tissue can be done with short pulses with a predetermined amount of energy. Alternatively, the delivery of energy may be limited by measuring the skin temperature and shutting off the energy when a predetermined temperature level has been reached.

The sufficient bulk heating level may be determined by skin inflammatory response appearing as erythema and edema.

The fractional treatment is applied to the same area creating a matrix of small coagulation and/or ablation zones. The small coagulation zones can be created by scanning a focused laser beam over the treatment zone or by delivering multiple laser beams (e.g., of different wavelengths) simultaneously. The laser parameters may be optimized to create coagulation and ablation of tissue. The range of wavelengths used for fractional treatment may be from 1500 nm to 10600 nm. The most popular lasers for fractional laser are CO₂ laser, erbium: YAG laser, erbium glass fiber laser, thulium laser.

The energy for each micro-spot may be in the range of 10 mJ up to 200 mJ and the diameter of coagulation zone may be in the range of 40 microns to 500 microns.

Ablation/coagulation depth depends on treatment objectives and may range from 100 microns up to a few millimeters.

Alternatively, other types of energy can be used for fractional treatment, including micro-needling, focused ultrasound.

The treatment that combines bulk heating and fractional ablation can be used for collagen shrinkage, wrinkle treatment, acne scar treatment, skin tightening and stretch mark treatment, and other applications requiring collage remodeling.

There is provided in accordance with a non-limiting embodiment of the invention a method for fractional tissue treatment including using a first energy source to heat a skin treatment zone to a sub-necrotic temperature level, and using a second energy source to apply fractional energy to the skin treatment zone to create a matrix of ablation and/or coagulation micro-zones.

In accordance with a non-limiting embodiment of the invention the first energy source is a laser that emits energy to a skin penetration depth greater than 0.5 mm.

In accordance with a non-limiting embodiment of the invention the first energy source is a laser, a vertical-cavity surface-emitting laser, a filament lamp or a flash lamp.

In accordance with a non-limiting embodiment of the invention the first energy source is an RF energy source.

In accordance with a non-limiting embodiment of the invention the first energy source creates tissue bulk heating up to a temperature in a range of 40° C. to 50° C.

In accordance with a non-limiting embodiment of the invention the second energy source is an RF energy source.

In accordance with a non-limiting embodiment of the invention the second energy source is a fractional laser.

In accordance with a non-limiting embodiment of the invention the second energy source includes a micro-needling device.

In accordance with a non-limiting embodiment of the invention the second energy source is a focused ultrasound device.

In accordance with a non-limiting embodiment of the invention the second energy source creates tissue ablation to a depth of 0.1 mm up to 10 mm.

In accordance with a non-limiting embodiment of the invention the second energy source creates tissue coagulation to a depth of 0.1 mm up to 10 mm.

There is provided in accordance with a non-limiting embodiment of the invention a device for fractional tissue treatment including one or more RF electrodes connected to a first RF energy source, configured to heat a tissue zone to a sub-necrotic temperature, micro-needle electrodes arranged for insertion into the tissue zone and coupled to a second RF source and operative to create a matrix of coagulation and/or ablation zones in the tissue zone, and a microcontroller coupled to the first and second RF energy sources, the one or more RF electrodes and the micro-needle electrodes, configured to control heating and micro-needles treatment to be delivered essentially simultaneously.

In accordance with a non-limiting embodiment of the invention the microcontroller is configured to control the heating to be delivered prior to the micro-needling treatment.

In accordance with a non-limiting embodiment of the invention the microcontroller is configured to control the heating to be delivered after to the micro-needling treatment.

In accordance with a non-limiting embodiment of the invention the microcontroller is configured so that a pulse of heating overlaps with an RF pulse delivered to the micro-needle electrodes.

In accordance with a non-limiting embodiment of the invention the micro-needle electrodes are inserted into the tissue zone to a depth from 0.1 mm up to 10 mm.

There is provided in accordance with a non-limiting embodiment of the invention a device for fractional tissue treatment including a heat source, configured to heat a tissue zone to a sub-necrotic temperature, a fractional laser operative to create a matrix of coagulation and/or ablation zones in the tissue zone, and a microcontroller coupled to the heat source and the fractional laser, configured to control heating and fractional laser treatment to be delivered essentially simultaneously.

In accordance with a non-limiting embodiment of the invention the heat source includes one or more RF electrodes connected to an RF energy source.

In accordance with a non-limiting embodiment of the invention the heat source includes one or more sources of optical energy.

In accordance with a non-limiting embodiment of the invention the fractional laser is an Er: YAG laser, a CO₂ laser, an Er: Glass laser or a thulium laser.

In accordance with a non-limiting embodiment of the invention the fractional laser includes a scanner for scanning a laser beam over a treatment area.

In accordance with a non-limiting embodiment of the invention the fractional laser includes a splitter operative to a laser beam from a laser into multiple micro-beams applied to a treatment area.

### BRIEF DESCRIPTION OF DRAWINGS

The present invention will be understood and appreciated more fully from the following detailed description, taken in conjunction with the drawings in which:

FIG. 1 is a simplified illustration of skin with bulk heating and fractional ablation.

FIG. 2 is a simplified illustration of one device with two combined laser beams.

FIG. 3 is a simplified illustration of one device with fractional laser and non-laser light source.

FIG. 4 is a simplified illustration of one device combining fractional laser and RF bulk heating.

FIG. 5 is a simplified illustration of one device combining RF micro-needling and bipolar RF bulk heating.

FIG. 6 is a simplified illustration of skin with bulk heating and fractional heating using micro-needling device.

### DETAILED DESCRIPTION

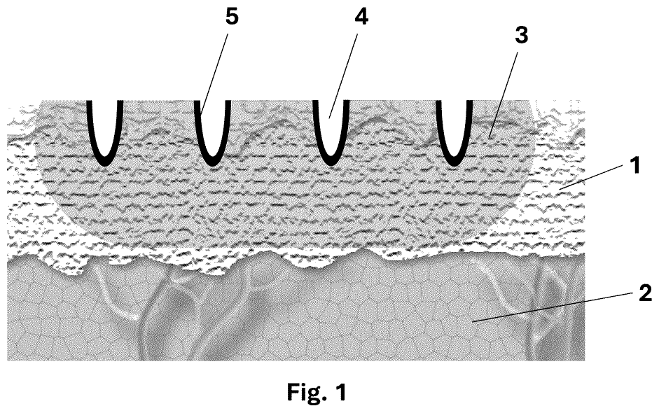

Reference is now made to FIG. 1, which illustrates a method of treatment, which may include creating a bulk sub-necrotic heating zone 3 in dermis 1 and creating ablation craters 4 surrounded or lined by a coagulation zone 5 in the same skin zone. In some treatments the depth of treatment can be extended into the subcutaneous fat 2.

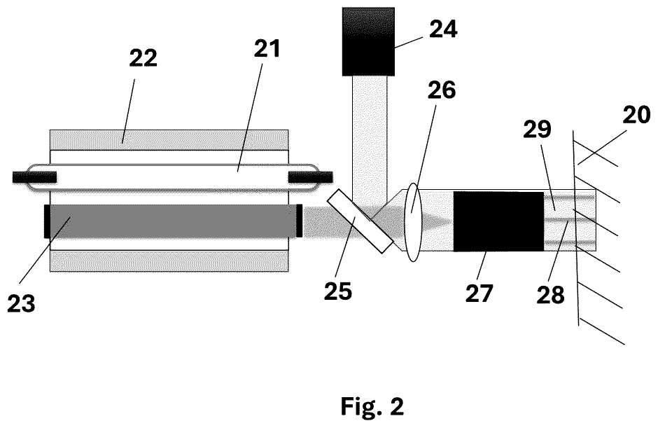

Reference is now made to FIG. 2, which illustrates a device for fractional and bulk heating including a flash lamp 21 (e.g., pumped laser). Light generated from flash lamp 21 may be reflected by a reflector 22 towards a crystal laser medium 23 coated with one or more lasing materials, thereby emitting a first laser beam towards a beam combiner 25 and a lens 26. The laser beam is fractionated by an optical element 27 so that a matrix of micro-beams 28 is propagated toward the skin surface 20. The laser medium 23 can be Erbium: YAG laser or other near infrared laser with strong absorption in tissue. The optimal energy per micro-beam is in the range of 10 mJ to 200 mJ. The diameter of micro-beam may be from 40 microns to 500 microns. The pulse width should not exceed 30 ms to minimize heat dissipation and maximize ablation and coagulation effect. The number of fractional zones can be varied from 10 up to 150 per square centimeter depending on size of coagulation crater. Ablation depth is controlled by energy per micro-beam. The optimal ablation depth is in the range 0.2 up to 2 mm.

Bulk heating may be done by energy of a diode laser 24, which may be adjusted to create tissue heating from 40° C. to 47° C. The power of the diode laser may be 5 W to 40 W and energy may be delivered in pulses with a pulse width from 100 ms up to 3 seconds. The longer pulse width will reduce treatment speed significantly.

The diode laser 24 may generate a laser beam in the range of 700 nm to 1500 nm, which is combined with the fractional laser using the beam combiner 25 and irradiating an essentially uniform second beam 29 to the skin surface 20.

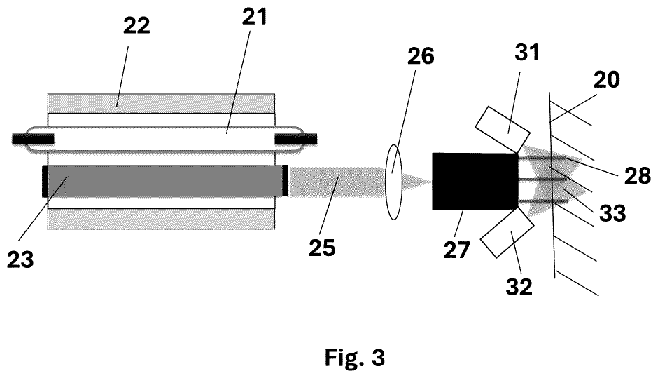

FIG. 3 illustrates a different version in which the fractional flash lamp pumped fractional laser is combined with non-laser light sources 31 and 32 which irradiate the same skin surface 20 with light beams 33 heating skin uniformly. Light emitting diodes or lamp can be used for skin bulk heating.

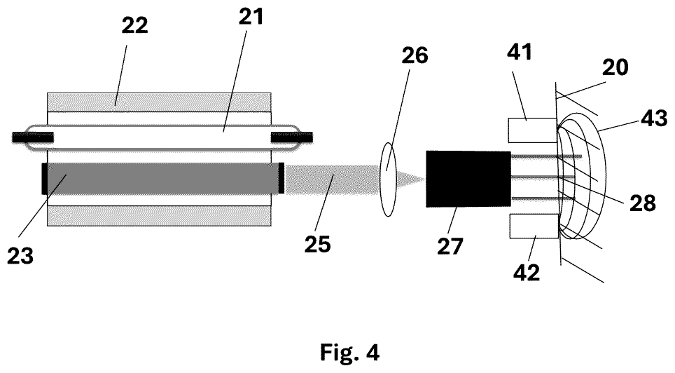

FIG. 4 illustrates a different version in which the fractional flash lamp pumped fractional laser is combined with RF heating. RF energy may be applied between two electrodes 41 and 42 coupled to the skin surface 20. RF current 43 flows between the electrodes 41 and 42 through the tissue. The bulk heating created by RF current 43 overlaps with small ablation zones created by micro-beams 28. The optimal RF frequency may be from 0.3 MHz up to 6 MHz. The RF power may be varied from 5 W up to 500 W and delivered energy can be controlled by RF pulse width or RF amplitude. A temperature sensor can be embedded into the device to monitor skin surface temperature. RF energy delivery can be adjusted according to the measured temperature.

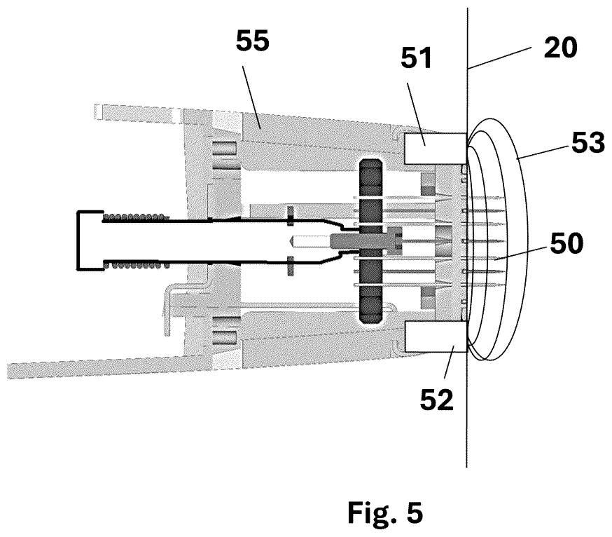

Referring to FIG. 5, the micro-needling device 55 is applied to the skin surface 20 and needles 50 are pushed into the skin to the predetermined depth. Part of the needle 50 can be coated with an electrically insulating material, whereas the sharp end of the needle 50 is conductive. RF energy is delivered through the conductive end of the needles 50 creating fractional ablation inside the tissue. The RF energy may be delivered in a pulsed manner and RF energy per needle may be in the range of 5 mJ up to 500 mJ. RF frequency may be in the range of 0.3 MHz up to 6 MHz. The bulk heating may be created by RF energy applied between two electrodes 51 and 52. The RF current 53 flows through the same tissue volume.

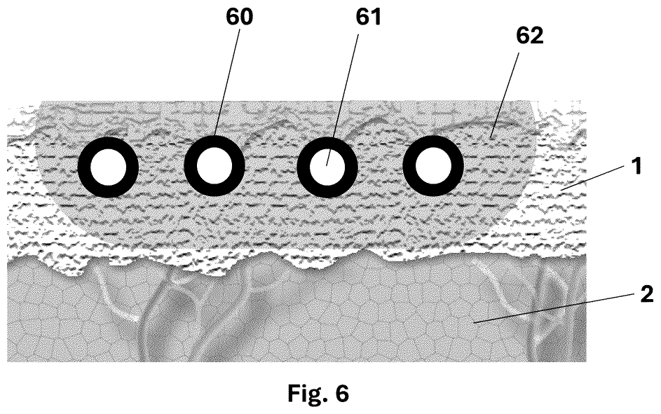

FIG. 6 shows ablation zones 61 and coagulation zones 62 inside the dermis 1. Bulk heating zone 62 is overlapped with fractional treatment zones. For some treatment the treatment depth can be extended into the sub-dermal area 2.

The term “approximately” or “about” is defined as plus or minus 10%.

1. A method for fractional tissue treatment comprising:
using a first energy source to heat a skin treatment zone to a sub-necrotic temperature level; and
using a second energy source to apply fractional energy to said skin treatment zone to create a matrix of ablation and/or coagulation micro-zones.

2. The method according to claim 1, wherein said first energy source is a laser that emits energy to a skin penetration depth greater than 0.5 mm.

3. The method according to claim 1, wherein said first energy source is laser, light emitting diode, a vertical-cavity surface-emitting laser, a filament lamp or a flash lamp.

4. The method according to claim 1, wherein said first energy source is an RF energy source.

5. The method according to claim 1, wherein said first energy source creates tissue bulk heating up to a temperature in a range of 40° C. to 50° C.

6. The method according to claim 1, wherein said second energy source is an RF energy source.

7. The method according to claim 1, wherein said second energy source is a fractional laser.

8. The method according to claim 1, wherein said second energy source comprises a RF micro-needling device.

9. The method according to claim 1 wherein said second energy source is a focused ultrasound device.

10. The method according to claim 1, wherein said second energy source creates tissue ablation to a depth of 0.1 mm up to 10 mm.

11. The method according to claim 1, wherein said second energy source creates tissue coagulation to a depth of 0.1 mm up to 10 mm.

12. A device for fractional tissue treatment comprising:
one or more RF electrodes connected to a first RF energy source, configured to heat a tissue zone to a sub-necrotic temperature;
micro-needle electrodes arranged for insertion into said tissue zone and coupled to a second RF source and operative to create a matrix of coagulation and/or ablation zones in said tissue zone; and
a microcontroller coupled to said first and second RF energy sources, said one or more RF electrodes and said micro-needle electrodes, configured to control heating and micro-needles treatment to be delivered essentially simultaneously.

13. The device according to claim 12, wherein said microcontroller is configured to control the heating to be delivered prior to the micro-needling treatment.

14. The device according to claim 12, wherein said microcontroller is configured to control the heating to be delivered after to the micro-needling treatment.

15. The device according to claim 12, wherein said microcontroller is configured so that a pulse of heating overlaps with an RF pulse delivered to the micro-needle electrodes.

16. The device according to claim 12, wherein said micro-needle electrodes are inserted into the tissue zone to a depth from 0.1 mm up to 10 mm.

17. A device for fractional tissue treatment comprising:
a heat source, configured to heat a tissue zone to a sub-necrotic temperature;
a fractional laser operative to create a matrix of coagulation and/or ablation zones in said tissue zone; and
a microcontroller coupled to said first heat source, and said fractional laser, configured to control heating and fractional laser treatment to be delivered essentially simultaneously.

18. The device according to claim 17, wherein said heat source comprises a one or more RF electrodes connected to an RF energy source.

19. The device according to claim 17, wherein said heat source comprises one or more sources of optical energy.

20. The device according to claim 17, wherein said fractional laser is an Er: YAG laser, a CO₂ laser, an Er: Glass laser or a thulium laser.

21. The device according to claim 17, wherein said fractional laser comprises a scanner for scanning a laser beam over a treatment area.

22. The device according to claim 17, wherein said fractional laser comprises a splitter operative to a laser beam from a laser into multiple micro-beams applied to a treatment area.
